(19) United States
(12) Patent Application Publication
(10) Pub. No.: US 2025/0303160 A1
(43) Pub. Date: Oct. 2, 2025

(54) SYSTEMS AND METHODS FOR GENERATING LOW-FREQUENCY STIMULATION

(71) Applicant: Creation Energy Technologies, LLC, Coeur d'Alene, ID (US)

(72) Inventors: Craig Coad, Coeur d'Alene, ID (US); Frank Braillard, Coeur d'Alene, ID (US)

(21) Appl. No.: 19/097,689

(22) Filed: Apr. 1, 2025

Related U.S. Application Data
(60) Provisional application No. 63/573,251, filed on Apr. 2, 2024.

(51) Int. Cl. A61N 1/36; A61N 1/04; A61N 1/08; A61N 1/10; A61N 1/32

(52) U.S. Cl. CPC A61N 1/36034; A61N 1/0476; A61N 1/08; A61N 1/10; A61N 1/328

(57) ABSTRACT

A system comprises a housing; an electrical circuit disposed at least partially within the housing, and an output element. The electrical circuit is configured to generate an electrical signal having a frequency of about 3.8 Hz, about 6 Hz, about 14 Hz, about 44 Hz, about 65 Hz, about 66 Hz, about 74 Hz, about 222 Hz, about 344 Hz, about 984 Hz, or any combination thereof. The output element is connected to the electrical circuit and is configured to output the electrical signal. The electrical signal can be transmitted to a user in response to a portion of the user contacting the output element, which aids in stimulating cellular regeneration in the user. The output element can include a plurality of separate output elements, and the electrical circuit can be sequentially connected to the output elements according to a connection frequency.

### CROSS REFERENCE TO RELATED APPLICATIONS

This application claims priority to and the benefit of U.S. Provisional Application No. 63/573,251 filed Apr. 2, 2024, which is hereby incorporated by reference herein in its entirety.

### TECHNICAL FIELD

The present disclosure relates generally to systems and methods for generating stimulation to be applied to a user, and more particularly, to systems and methods for generating low-frequency electrical and optical stimulation to be applied to a user.

### BACKGROUND

Current systems and methods for applying various types of stimulation to users are generally unsuitable or ineffective. Thus, new systems and methods are needed for generating and applying stimulation to users.

### SUMMARY

According to some implementations of the present disclosure, a system includes a housing; an electrical circuit disposed at least partially within the housing, the electrical circuit being configured to generate an electrical signal having a frequency of less than 1,000 Hz, and a voltage of at least 100 volts; and an output element electrically coupled to the electrical circuit and configured to output the electrical signal. The frequency of the electrical signal may be about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

According to some implementations of the present disclosure, a system comprises a housing; an electrical circuit disposed at least partially within the housing, and an output element. The electrical circuit is configured to generate an electrical signal. The optical output element is configured to receive the electrical signal and output an optical signal. The frequency of the optical signal may be about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

According to some implementations, a method comprises generating, via a low-frequency signal generator, a first initial signal having a target frequency; generating, via an amplifier that receives the first initial signal, a second initial signal having the target frequency and an increased amperage relative to the first initial signal; generating, via a high-frequency signal generator, a carrier signal having a carrier frequency that is greater than the target frequency; generating, via a step-up transformer that receives the second initial signal and the carrier signal, an amplitude-modulated signal having a voltage of at least 100 volts, the amplitude-modulated signal having an envelope corresponding to the target frequency; and generating, via an envelope detector that receives the amplitude-modulated signal, an electrical signal corresponding to the envelope of the amplitude-modulated signal and having the target frequency and a voltage of at least 100 volts.

According to some implementations, a method comprises generating a stimulation having a frequency of less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

According to some implementations, a method comprising generating, via an electrical circuit, an electrical signal having a voltage of at least 100 volts; and connecting the electrical circuit to an output element, such that the electrical signal is transmitted from the output element to a user in response to a portion of the user contacting the output element. The electrical signal aids in stimulating cellular regeneration in the user.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The disclosure, and its advantages and drawings, will be better understood from the following description of representative embodiments together with reference to the accompanying drawings. These drawings depict only representative embodiments, and are therefore not to be considered as limitations on the scope of the various embodiments or claims.

FIG. 1 is a first system for generating an electrical stimulation, according to aspects of the present disclosure.

FIG. 2A is a first implementation of the internal components of the system of FIG. 1, according to aspects of the present disclosure.

FIG. 2B is a second implementation of the internal components of the system of FIG. 1, according to aspects of the present disclosure.

FIG. 3 is a second system for generating an electrical stimulation, according to aspects of the present disclosure.

FIG. 4 is a system for generating an optical stimulation, according to aspects of the present disclosure.

FIG. 5 is a flow chart of a method for generating an electrical stimulation, according to aspects of the present disclosure.

FIG. 6 is a conducting plate array comprising a plurality of conducting plates arranged in a spiral pattern, according to aspects of the present disclosure.

While the present disclosure is susceptible to various modifications and alternative forms, specific implementations and embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

Disclosed herein are systems and methods for generating low-frequency stimulations (e.g., electrical signals, optical signals, etc.), and outputting such stimulations in a manner allowing for the stimulation to be applied and/or transferred to a human. These stimulations provide a number of health and wellness benefits, including aiding in the cellular regeneration process following damage or trauma, and in restoring regenerative vitality.

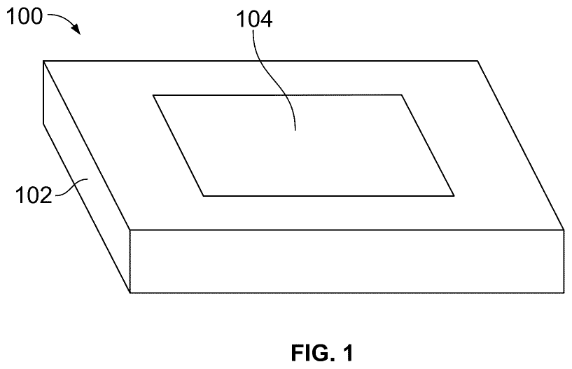

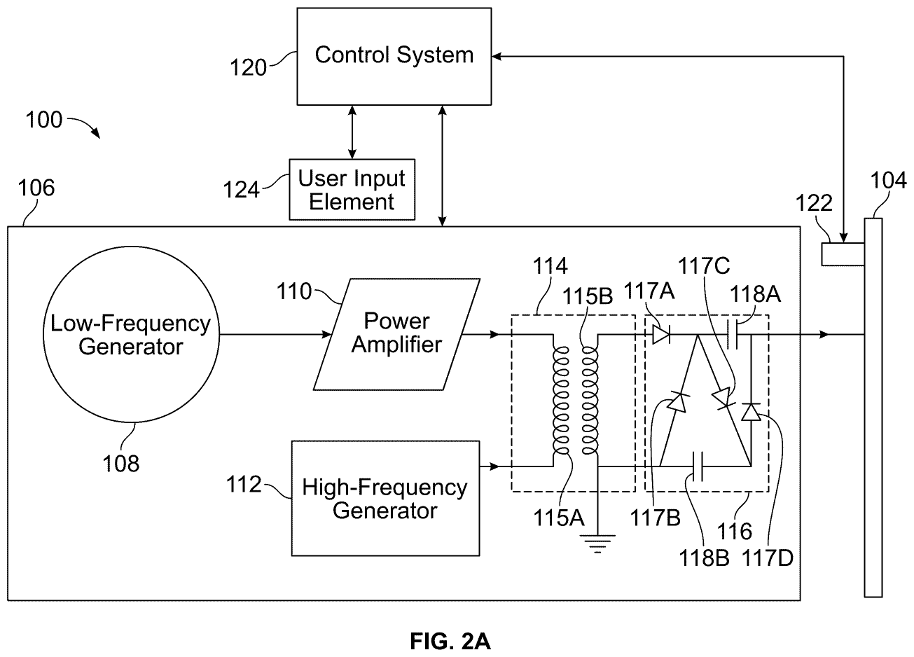

FIG. 1 illustrates a system 100 used for providing low-frequency stimulation to a user. The system 100 includes a housing 102 and a glass plate 104 mounted in the housing 102. The housing 102 in this implementation generally forms a box that includes internal component mounted therein. The glass plate 104 has a generally flat surface that is externally accessible by the user. The housing 102 generally includes an electrical circuit 106 (FIGS. 2A and 2B) disposed therein. The system 100 can be used to generate a stimulation (e.g., electrical stimulation, such as a static or moving electric field), which can be output by the glass plate 104. Specifically, the electrical circuit 106 is configured to generate an electrical signal that is applied to the glass plate 104. In FIG. 1, the glass plate 104 forms is the output element of the system 100, which is generally the component of the system 100 that can be used to apply the stimulation to the user. Other types of output elements can also be used however, such as a glass tube, a glass bulb or sphere, other insulating material, etc.

FIG. 2A illustrates a first implementation of the system 100 and shows the electrical circuit 106 and other components that are disposed within the housing 102. The electrical circuit 106 includes a low-frequency signal generator 108, an amplifier 110, a high-frequency signal generator 112, a step-up transformer 114, and a voltage multiplier 116. The electrical circuit 106 may include one or power-related components, such as a power supply configured to be connected to mains power and/or a battery. The power supply and/or the battery can be used to power the various components of the electrical circuit 106 and/or any of the other components that require power.

The low-frequency signal generator 108 is configured to generate a first initial electrical signal that has a target frequency. The target frequency can generally be any suitable frequency that is desired to be applied to the user. In some implementations, the frequency is generally any frequency that is less than or equal to about 1,000 hertz (Hz). In some implementations, the target frequency is between 3 hertz (Hz) and 5 Hz, and/or is about 3.8 Hz. In some implementations, the target frequency may be selected based for a specific use of the system 100. Any type of alternating signal can be generated, such as a sine wave, a cosine wave, a square wave, etc. Generally, the first initial signal will have a relatively low voltage and amperage. The amplifier 110 is electrically connected to the output of the low-frequency signal generator 108. The amplifier 110 receives the first initial signal from the low-frequency signal generator 108 and generates a second initial electrical signal by amplifying the first initial signal. The second initial signal will have an increased amperage relative to the first initial signal, but will generally have the same frequency as the first initial signal, i.e., the target frequency.

Separately from the low-frequency signal generator 108 and the amplifier 110, the high-frequency signal generator 112 is configured to generate a carrier electrical signal. The carrier signal will generally have a much high frequency than the target frequency of the first initial signal and the second initial signal, such as greater than 10 Hz.

The step-up transformer 114 has a primary winding 115A that is electrically connected to the outputs of both the amplifier 110 and the high-frequency signal generator 112. The step-up transformer 114 receives the second initial signal and the carrier signal, and generates an amplitude-modulated signal based on the two input signals at the secondary winding 115B. The amplitude-modulated signal will generally have a voltage of at least 100 volts, and in some implementations has a voltage of at least 2,000 volts. The amplitude-modulated signal will have a relatively high frequency that is generally equal to the frequency of the carrier signal. The amplitude-modulated signal will also have an envelope that is formed by the varying amplitude. This envelope will have an envelope frequency that is generally equal to the low frequency of the first initial signal and the second initial signal, such as between 3 Hz and 5 Hz and/or about 3.8 Hz. Thus, the step-up transformer 114 acts to modulate the amplitude of the carrier signal at a frequency that is generally equal to the target frequency. The result is an electrical signal having an envelope frequency equal to the target frequency, but with a much higher voltage than the first initial signal and the second initial signal.

The voltage multiplier 116 is electrically connected to the output of the step-up transformer 114, and receives the amplitude-modulated signal from the step-up transformer 114. The voltage multiplier 116 includes four diodes 117A-117D and two capacitors 118A and 118B.

The anode of the first diode 117A is connected to a first end of the secondary winding 115B. The cathode of the first diode 117A is connected to the cathode of the second diode 117B, the anode of the third diode 117C, and the first end of the first capacitor 118A. The anode of the second diode 117B is connected to a second end of the secondary winding 115B (which is grounded) and the first end of the second capacitor 118B. The cathode of the second diode 117B is connected to the cathode of the first diode 117A, the anode of the third diode 117C, and the first end of the first capacitor 118A. The anode of the third diode 117C is connected to the cathode of the first diode 117A, the cathode of the second diode 117B, and the first end of the first capacitor 118A. The cathode of the third diode 117C is connected to the second end of the second capacitor 118B and the anode of the fourth diode 117D. The anode of the fourth diode 117D is connected to the cathode of the third diode 117B and the second end of the second capacitor 118B. The cathode of the fourth diode 117D is connected to the second end of the first capacitor 118A.

The first end of the first capacitor 118A is connected to the cathode of the first diode 117A, the cathode of the second diode 117B, and the anode of the third diode 117C. The second end of the capacitor 118A is connected to the cathode of the fourth diode 117D. The first end of the second capacitor 118B is connected to the second end of the secondary winding 115B and the anode of the second diode 117B. The second end of the second capacitor 118B is connected to the cathode of the third diode 117C and anode of the fourth diode 117D. The connection between the second end of the first capacitor 118A and the cathode of the fourth diode 117D forms the output of the voltage multiplier 116, which is connected directly to the glass plate 104.

The voltage multiplier 116 is configured to generate the electrical signal that will be applied to the glass plate 104 from the envelope of the amplitude-modulated signal. The voltage multiplier 116 thus operates to detect and extract the envelope of the amplitude-modulated signal. Because the electrical signal generated by the voltage multiplier 116 is generated from the envelope of the amplitude-modulated signal, the electrical signal will have a frequency equal to the envelope, i.e., the target frequency. Moreover, the electrical signal will generally also have a voltage equal to the voltage of the amplitude-modulated signal, which is generally at least 100 volts.

The system 100 can further include a controller 120 and a sensor 122 disposed within the housing that controls operations of the electrical circuit 106 and/or other components. The controller 120 can cause the electrical circuit 106 to begin generating the electrical signal and to stop generating the electrical system. The sensor 122 is coupled to the glass plate 104 (or other output element) generates data indicative of external contact with the glass plate 104, such as contact between the glass plate 104 and human skin (e.g., a hand, a face, etc.). The controller 120 is configured to detect this external contact based on the data, and in response cause the electrical circuit 106 to generate the electrical signal, which is then transmitted to the glass plate 104 and the user. In some implementations, the controller 120 causes the electrical circuit 106 to generate the electrical signal substantially immediately after detecting this external contact, but in other implementations, the controller 120 causes the electrical circuit 106 to generate the electrical signal after a predetermined amount of time has passed following the detection of the external contact (e.g., about 1 second, about 5 seconds, about 10 seconds, etc.), to prevent inadvertent contact with the glass plate 104 from immediately activating the electrical circuit 106. Moreover, in some cases, the sensor 122 generates data indicative of the proximity of an object (such as the user's hand or face) to the glass plate 104, instead of or in addition to actual contact. The controller 120 can similarly cause the electrical circuit 106 to generate the electrical signal in response to the user being within a threshold proximity to the glass plate 123 (either immediately or after a predetermined amount of time). In some implementations, the system 100 further includes a user input element 124 communicatively coupled to the controller 120. The user input element 124 allows the user to provide any sort of desired input to the system 100, and may include a switch, a touchscreen, etc.

FIG. 2B illustrates a second implementation of the system 100, which includes generally the glass plate 104, same electrical circuit 106, controller 120, and sensor 122. However, the system 100 in FIG. 2B further includes a conducting plate 126 (made from copper and/or similar materials) that is electrically connected to both the output of the electrical circuit 106 (e.g., the output of the voltage multiplier 116) and the glass plate 104, which results in the entire glass plate 104 (or a large portion of the glass plate 104) being in contact with the conducting plate 126 to efficiently transmit the electrical signal to the glass plate 104.

In either implementation, the electrical circuit 106 operates to take a low-voltage signal at the target frequency and increase the voltage to at least 100 volts. The electrical signal that is applied to the glass plate 104 (either directly or via the conducting plate 126) can thus have a frequency that is between 3 Hz and 5 Hz, or about 3.8 Hz. The electrical signal can also have a voltage of at least 100 volts, at least 2,000 volts, or any other suitable voltage. A user can then place their hand and/or another body part onto the glass plate 104, such that the electrical signal is applied to the user. The glass plate 104 acts as an electrical insulator, such that the high voltage of the electrical signal (e.g., at least 100 volts) only results in a small current (e.g., 100 μA or less, between 1 μA and 20 μA, etc.) when applied to the user. Other types of insulators can also be used instead of the glass plate 104, such as a gas tube or a gas globe. Thus, the electrical signal can generally have any suitable voltage (e.g., at least 100 volts, at least 2,000 volts, etc.), so long as there is some insulator used to prevent the user from being subjected to dangerously high currents.

In some implementations, only the low-frequency generator 108 or only the high-frequency generator 112 are used. For example, depending on the desired frequency of the electrical signal, only one of the frequency generators may be needed. Moreover, while a specific implementation of the electrical circuit 106 is illustrated in FIGS. 2A and 2B, other variations of the electrical circuit 106 may be used that include additional components beyond those illustrated and/or that exclude some (or all) illustrated components. For example, any suitable type of frequency generator may be used. In another example, the power amplifier 110 is not used depending on the application. In a further example, a type of voltage multiplier different than the voltage multiplier 116 is used. Thus, generally any electrical circuit may be used to generate the electrical signal.

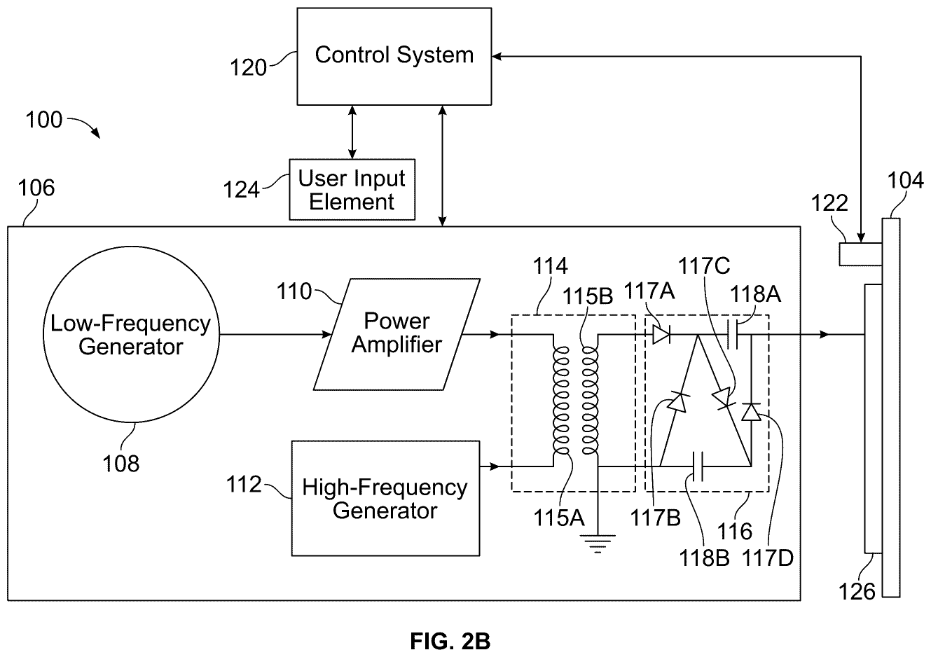

FIG. 3 shows a system 300, which is formed as a wand. System 300 is generally similar to system 100 except the electrical circuit 106 and the other components are positioned inside of the wand instead of the housing 102. The wand may include a thicker grip portion 302 that they user may grasp, and a thinner elongated rod 304 that terminates in an output element 306. The output element 306 could be a glass plate similar to the glass plate 104, a glass bulb or sphere, some other insulating material, etc. In general, the system 300 operates similarly to system 100, except that the user grasps the wand by the grip portion 302 and moves the output element 306 toward their skin (or other surface), instead of placing their hand on the glass plate 104. Further, the implementation illustrated in FIG. 3 is simply an example of a handheld system. In general, the electrical circuit 106 and any other required elements could be placed in generally any handheld housing to allow the user to hold the system and apply the stimulation to their skin.

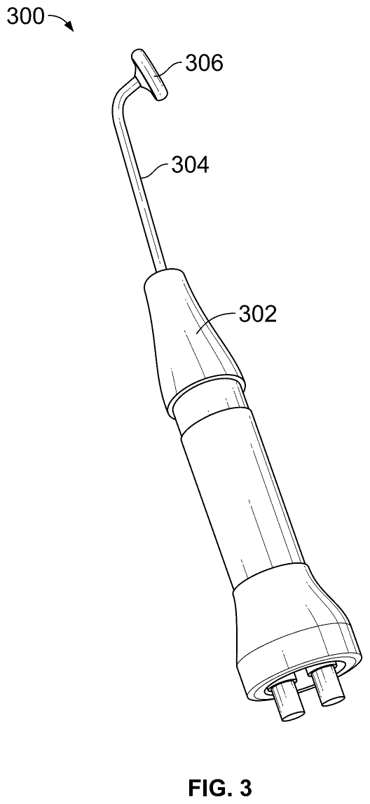

FIG. 4 shows a system 400 that can be used to generate and apply an optical signal to the user. The system 400 includes the control system 120, a low-frequency signal generator 402 (which may be the same as or similar to the low-frequency signal generator 108) and an optical output element 404. The low-frequency signal generator 402 generates an electrical signal which can then be transmitted to the optical output element 404 to drive the optical output element 404 and generate the optical signal. The optical output element can be a light-emitting array that includes one or more lasers, one or more LEDs, or both. However, other types of output elements can also be used in the system 400, such as magnetic coils. Generally, the optical signal (or whatever signal that is output by the output element) can have a frequency similar to that of the electrical signal output by the system 100. The electrical signal generated by the low-frequency signal generator 402 may have an identical frequency as the optical signal, or a different frequency.

In the illustrated implementation, the system 400 further includes a power amplifier 406, which amplifies the electrical signal generated by the low-frequency signal generator 402 before sending the amplified electrical signal to the optical output element 404, to thereby amplify the intensity of the optical signal. In other implementations however, the system 400 does not include the power amplifier 406.

The components of system 400 could be placed in a housing similar to the housing 102 of system 100 (e.g., a rectangular or square box), but could also be placed within the housing 302 of the wand 300. Moreover, system 400 may include a component covering the optical output element 404, such that this component is positioned between the optical output element 404 and the user's skin when in use. This component could include a glass plate similar to the glass plate 104, but other components can also be used. Moreover, similar to system 100 and system 300, system 400 can include a controller that controls the operation of the various components of system 400. The system 400 may also include a sensor to detect contact and/or proximity, in order to trigger the generation of the optical signal.

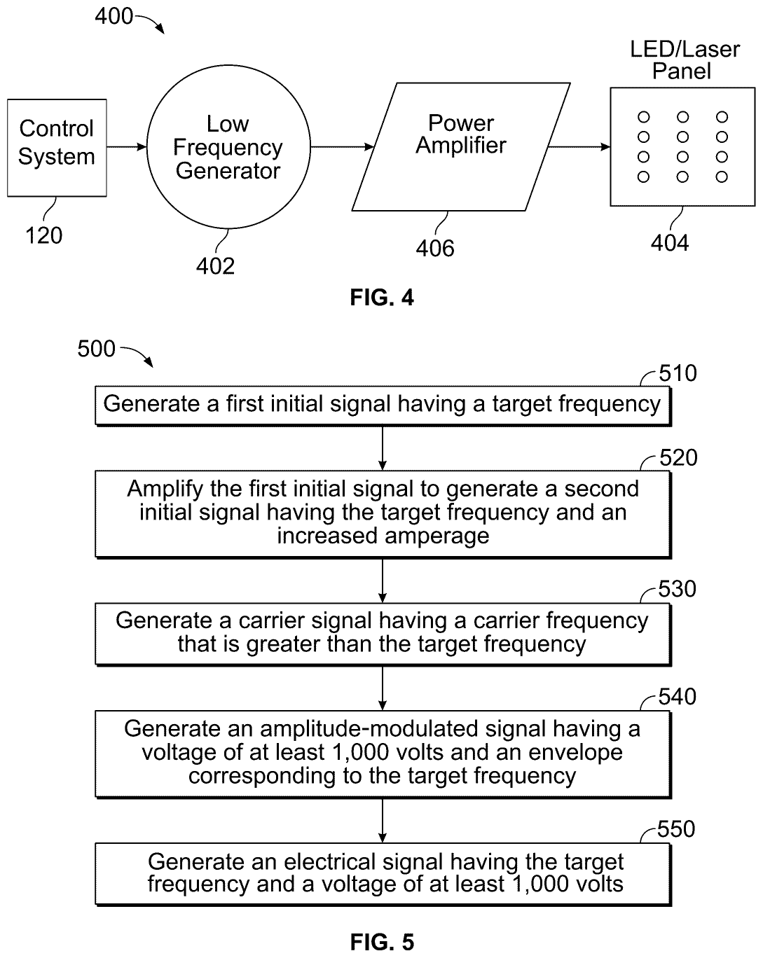

FIG. 5 shows a flow chart of a method 500 for generating an electrical stimulation. Generally, a controller having one or more processors (such as controller 120) is configured to carry out the steps of method 500. A memory device can be used to store machine-readable instructions that are executed by the controller to carry out the steps of method 500. The memory device can also store any type of data utilized in the steps of method 500. Generally, method 500 can be implemented using a system (such as systems 100, 300, or 400) that includes the controller and the memory device.

Step 510 of method 500 includes generating, via a low-frequency signal generator (such as the low-frequency signal generator 108), a first initial signal having a target frequency. Step 520 includes generating, via an amplifier that receives the first initial signal (such as the amplifier 110), a second initial signal having the target frequency and an increased amperage relative to the first initial signal. Step 530 includes generating, via a high-frequency signal generator (such as the high-frequency signal generator 112), a carrier signal having a carrier frequency that is greater than the target frequency. Step 540 includes generating, via a step-up transformer that receives the second initial signal and the carrier signal (such as the step-up transformer 114), an amplitude-modulated signal having a voltage of at least 100 volts. The amplitude-modulated signal has an envelope corresponding to the target frequency. Step 550 includes generating, via a voltage multiplier that receives the amplitude-modulated signal (such as the voltage multiplier 116), an electrical signal corresponding to the envelope of the amplitude-modulated signal and having the target frequency and a voltage of at least 100 volts.

The application of the various stimulations disclosed herein (e.g., high-voltage and low-frequency electrical signals, low-frequency optical signals, etc.) directs energy into the human body, and aids in ensuring proper cellular regeneration and performance. The use of low frequencies maximizes the uptake and utilization of the energy, without subjecting users to any deleterious electromagnetic frequencies.

As noted herein, any suitable frequency can be used for the stimulation. In some cases, the frequency is chosen based on the different purpose of the stimulation. For example, if the stimulation is used for wrinkle reduction, the frequency may be between about 60 Hz and about 70 Hz, between about 70 Hz and about 80 Hz, between about 60 Hz and about 80 Hz, about 65 Hz, about 74 Hz, between about 950 Hz and about 1,000 Hz, between about 980 Hz and about 990 Hz, about 984 Hz, or between about 60 Hz and about 990 Hz.

In another example, if the stimulation is used for tissue inflammation reduction, the frequency may be less than or equal to about 10 Hz, between about 40 Hz and about 50 Hz, between about 40 Hz and about 70 Hz, about 6 Hz, about 44 Hz, about 66 Hz, or less than or equal to 70 Hz.

In another example, if the stimulation is used for skin spot mitigation, the frequency may be between about 10 Hz and about 20 Hz, about 14 Hz, between about 200 Hz and about 250 Hz, between about 220 Hz and about 230 Hz, about 222 Hz, between about 300 Hz and about 350 Hz, between about 340 Hz and about 350 Hz, about 344 Hz, or between about 10 Hz and about 350 Hz.

In another example, if the stimulation is used for ache healing, the frequency may be between about 10 Hz and about 20 Hz, about 13 Hz, between about 30 Hz and about 40 Hz, between about 10 Hz and about 40 Hz, about 32 Hz, between about 60 Hz and about 70 Hz, between about 30 Hz and about 70 Hz, between about 10 Hz and about 70 Hz, about 63 Hz, or between about 10 Hz and about 70 Hz.

In another example, if the stimulation is used for scar tissue mitigation and healing, the frequency may be less than or equal to about 10 Hz, about 6 Hz, between about 660 Hz and about 670 Hz, between about 600 Hz and about 700 Hz, about 666 Hz, between about 790 Hz and about 800 Hz, between about 700 Hz and about 800 Hz, between about 600 Hz and about 800 Hz, about 796 Hz, or less than or equal to about 800 Hz

In some implementations, the frequency of the stimulation periodically alternates between two or more different frequencies. This can enhance the effectiveness of the stimulation applied to the user. If the stimulation is used for wrinkle reduction, the frequency of the stimulation may alternate between at least two different frequencies selected from the frequencies and ranges disclosed herein. In one specific example, the frequency may cycle between a first frequency between about 60 Hz and about 70 Hz, a second frequency between about 70 Hz and about 80 Hz, and a third frequency between about 980 Hz and about 990 Hz. In another specific example, the frequency may cycle between a first frequency of about 65 Hz, a second frequency of about 74 Hz, and a third frequency of about 984 Hz.

If the stimulation is used for tissue inflammation reduction, the frequency of the stimulation may cycle between at least two different frequencies selected from the frequencies and ranges disclosed herein. In one specific example, the frequency may cycle between a first frequency that is less than or equal to about 10 Hz, a second frequency between about 40 Hz and about 50 Hz, and a third frequency between about 60 Hz and about 70 Hz. In another specific example, the frequency may cycle between a first frequency of about 6 Hz, a second frequency of about 44 Hz, and a third frequency of about 66 Hz.

If the stimulation is used for skin spot mitigation, the frequency of the stimulation may cycle between at least two different frequencies selected from the frequencies and ranges disclosed herein. In one specific example, the frequency may cycle between a first frequency that is between about 10 Hz and about 20 Hz, a second frequency between about 220 Hz and about 230 Hz, and a third frequency between about 340 Hz and about 350 Hz. In another specific example, the frequency may cycle between a first frequency of about 14 Hz, a second frequency of about 222 Hz, and a third frequency of about 344 Hz.

If the stimulation is used for ache healing, the frequency of the stimulation may cycle between at least two different frequencies selected from the frequencies and ranges disclosed herein. In one specific example, the frequency may cycle between a first frequency that is between about 10 Hz and about 20 Hz, a second frequency between about 30 Hz and about 40 Hz, and a third frequency between about 60 Hz and about 70 Hz. In another specific example, the frequency may cycle between a first frequency of about 13 Hz, a second frequency of about 32 Hz, and a third frequency of about 64 Hz.

If the stimulation is used for scar tissue mitigation and healing, the frequency of the stimulation may cycle between at least two different frequencies selected from the frequencies and ranges disclosed herein. In one specific example, the frequency may cycle between a first frequency that is less than or equal to about 10 Hz, a second frequency between about 660 Hz and about 670 Hz, and a third frequency between about 790 Hz and about 800 Hz. In another specific example, the frequency may cycle between a first frequency of about 6 Hz, a second frequency of about 666 Hz, and a third frequency of about 796 Hz.

In some implementations, the frequency of the stimulation is user selectable, for example via the user input element 124. The user may be able to select a specific frequency, a specific range of frequencies, etc. The user may also be able to select between different possible applications of the stimulation (e.g., wrinkle reduction, tissue inflammation reduction, skin spot mitigation, etc.), and the system can automatically select the correct frequency or frequency range. The system may also automatically select the multiple frequencies that are cycled through depending on the user's selected application.

While the present disclose contemplates the application of the stimulation to the user's skin via direct contact with the user's skin, in some implementations, the stimulation is applied by positioning the output element (e.g. the glass plate 104, the output element 306, the optical output element 404, etc.) in close proximity to the user's skin, without contacting the user's skin. For example, the user could place their hands in close proximity to the glass plate 104 in the system 100. In another example, the user could move the output element 306 in close proximity to skin to which the stimulation is to be applied.

In some implementations, a health-promoting substance can be applied to the output element and/or to the user's skin to enhance the effectiveness of the stimulation. For example, the health-promoting substance could be an essential oil. In some implementations, the health-promoting substance is applied first to the skin, and then the stimulation is applied (for example by placing the user's hands on the glass plate 104, by moving the output element 306 of the wand to the user's skin, etc.). In other implementations, the health-promoting substance is applied to the output element. In some of these implementations, the health-promoting substance is applied directly to the output element. In other implementations, the system may include a reusable or discardable cover that can be soaked in the health-promoting substance (or otherwise have the health-promoting substance applied thereto), and then attached to the output element.

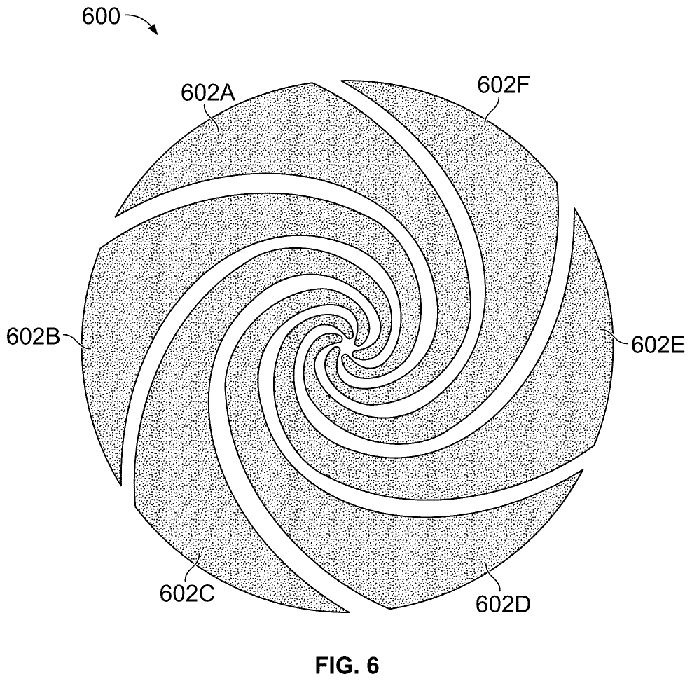

In some implementations, a moving or spinning stimulation can be created including multiple different conducting plates that are charged in sequential order. FIG. 6 illustrates a conducting plate array 600 that includes six separate conducting plates 602A-602F arranged in a spiral pattern. By outputting the electrical signal at the conducting plates 602A-602F in sequential order, the stimulation is applied in a spinning configuration. Thus, referring to the implementation of system 100 shown in FIG. 2B, instead of a single conducting plate 126 electrically connected to the output of the voltage multiplier 116, system 100 could include the spiral conducting plate array 600, which each conducting plate 602A-602F electrically connected to the output of the voltage multiplier 116. By connecting each of the conducting plates to the output of the voltage multiplier 116 individually in a sequence, the stimulation is applied in a spinning configuration. In some cases, the electrical circuit 106 may include a multi-way switch that is able to electrically connect only one of the conducting plates 602A-602F at a time to the output of the voltage multiplier 116. The multi-way switch can be controlled by the controller 120 to sequentially connect the conducting plates.

In some implementations, the controller 120 can control the frequency at which the electrical circuit is connected to the conducting plates, which is referred to herein as the connection frequency. In some implementations, the electrical stimulation itself has a frequency, and is sequentially applied to the different conducting plates at a connection frequency. In other implementations, a steady-state or near steady-state electrical stimulation is generated (for example by using the low-frequency generator 108 to generate a steady-state or near steady-state), and is sequentially applied to the different conducting plates at a specific connection frequency.

The connection frequency according to which electrical circuit is connected to the plurality of conducting plates can be any suitable frequency. In some implementations, any frequency which is discussed herein as a possible frequency for the electrical stimulation itself can additionally or alternatively be the connection frequency at which the electrical circuit and connected to the conducting plates and such stimulation (or a steady-state stimulation) is sequentially applied to the conducting plates. For example, in some cases if the stimulation is used for wrinkle reduction, the connection frequency at which the stimulation alternates between individual conducting plates can be between about 60 Hz and about 70 Hz, between about 70 Hz and about 80 Hz, between about 60 Hz and about 80 Hz, about 65 Hz, about 74 Hz, between about 950 Hz and about 1,000 Hz, between about 980 Hz and about 990 Hz, about 984 Hz, or between about 60 Hz and about 990 Hz.

In some implementations, the electrical stimulation can be output at multiple of the conducting plates 602A-602F at the same time. For example, the electrical stimulation could be applied to a specific pair of the conducting plates 602A-602F, and then alternated between different pairs of the conducting plates 602A-602F. In some implementations, the conducting plates are not arranged in a spiral like the conducting plates 602A-602F shown in FIG. 6, but instead are arranged in other shapes, such as a circular array, a triangular array, etc.

In some implementations, the principles of the spiral conducting plate array 600 and/or other shapes of conducting arrays can also be used with the system 400 that generates an optical signal to be applied to a user. For example, instead of a single LED or laser panel 404, an optical array formed from a series of LED or laser panels can be used. This optical array could be arranged in a spiral shapes like the conducting array 600 and the conducting plates 602A-602F, but could be arranged in other shapes as well.

In some implementations, aspects of the present disclosure can be applied to a method using two (or more) users. For example, two instances of the system 100 may be provided, with each user placing a hand on one instance of the system 100 to receive the electrical stimulation. The first user can then move their unused hand over the skin of the second user, so as to deliver electrical stimulation to the second user.

Thus, the features disclosed herein can be used to provide numerous health and wellness benefits to users.

Generally, any of the methods disclosed herein can be implemented using a system having a control system with one or more processors, and a memory device storing machine-readable instructions. The control system can be coupled to the memory device, and methods can be implemented when the machine-readable instructions are executed by at least one of the processors of the control system. The methods can also be implemented using a computer program product (such as a non-transitory computer readable medium) comprising instructions that when executed by a computer, cause the computer to carry out the steps of the methods.

One or more elements or aspects or steps, or any portion(s) thereof, from one or more of any of claims below can be combined with one or more elements or aspects or steps, or any portion(s) thereof, from one or more of any of the other claims or combinations thereof, to form one or more additional implementations and/or claims of the present disclosure.

### Alternative Implementations

Alternative Implementation 1. A system comprising: a housing; an electrical circuit disposed at least partially within the housing, the electrical circuit being configured to generate an electrical signal having a frequency of less than 1,000 Hz, and a voltage of at least 100 volts; and an output element electrically coupled to the electrical circuit and configured to output the electrical signal.

Alternative Implementation 2. The system of Alternative Implementation 1, wherein the frequency of the electrical signal is less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

Alternative Implementation 3. The system of Alternative Implementation 2, wherein the frequency of the electrical signal is about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

Alternative Implementation 4. The system of any one of claims 1 to 3, wherein the voltage of the electrical signal is at least 2,000 volts.

Alternative Implementation 5. The system of any one of claims 1 to 4, wherein the electrical signal has an amperage of between 1 μA and 20 μA.

Alternative Implementation 6. The system of any one of claims 1 to 5, wherein the frequency of the electrical signal is user-selectable.

Alternative Implementation 7. The system of any one of claims 1 to 6, wherein the frequency of the electrical signal is configured to periodically alternate between a plurality of different frequencies.

Alternative Implementation 8. The system of Alternative Implementation 7, wherein the plurality of different frequencies includes a first frequency between about 60 Hz and about 70 Hz, a second frequency between about 70 Hz and about 80 Hz, and a third frequency between about 980 Hz and about 990 Hz.

Alternative Implementation 9. The system of Alternative Implementation 8, wherein the first frequency is about 65 Hz, the second frequency is about 74 Hz, and the third frequency is about 984 Hz.

Alternative Implementation 10. The system of Alternative Implementation 7, wherein the plurality of different frequencies includes a first frequency that is less than or equal to about 10 Hz, a second frequency between about 40 Hz and about 50 Hz, and a third frequency between about 60 Hz and about 70 Hz.

Alternative Implementation 11. The system of Alternative Implementation 10, wherein the first frequency is about 6 Hz, the second frequency is about 44 Hz, and the third frequency is about 66 Hz.

Alternative Implementation 12. The system of Alternative Implementation 7, wherein the plurality of different frequencies includes a first frequency between about 10 Hz and about 20 Hz, a second frequency between about 220 Hz and about 230 Hz, and a third frequency between about 340 Hz and about 350 Hz.

Alternative Implementation 13. The system of Alternative Implementation 12, wherein the first frequency is about 14 Hz, the second frequency is about 222 Hz, and the third frequency is about 344 Hz.

Alternative Implementation 14. The system of any one of Alternative Implementations 1 to 13, wherein the output element is made from glass.

Alternative Implementation 15. The system of Alternative Implementation 14, wherein the output element includes an externally accessible glass plate mounted in the housing.

Alternative Implementation 16. The system of Alternative Implementation 14, wherein the output elements includes a glass bulb mounted in the housing or a glass sphere mounted in the housing.

Alternative Implementation 17. The system of Alternative Implementation 16, wherein the housing is sized to be graspable by a user.

Alternative Implementation 18. The system of any one of Alternative Implementations 1 to 17, wherein the electrical circuit includes: a low-frequency signal generator configured to generate a first initial signal having a target frequency; an amplifier electrically connected to the low-frequency signal generator, the current amplifier being configured to receive the first initial signal and generate a second initial signal having the target frequency and an increased amperage relative to the first initial signal; a high-frequency signal generator configured to generate a carrier signal having a carrier frequency that is greater than the target frequency; a step-up transformer electrically coupled to the current amplifier and the high-frequency signal generator, the step-up transformer being configured to receive the second initial signal and the carrier signal and output an amplitude-modulated signal having a voltage of at least 100 volts, the amplitude-modulated signal having an envelope frequency corresponding to the target frequency; and a voltage detector configured to receive the amplitude-modulated signal and generate the electrical signal from the envelope of the amplitude-modulated signal such that the frequency of the electrical signal is equal to the target frequency.

Alternative Implementation 19. The system of Alternative Implementation 18, wherein the target frequency is less than or equal to about 1,000 Hz.

Alternative Implementation 20. The system of Alternative Implementation 19, wherein the target frequency is less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

Alternative Implementation 21. The system of Alternative Implementation 20, wherein the target frequency is about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

Alternative Implementation 22. The system of any one of Alternative Implementations 18 to 20, wherein the low-frequency signal generator, the amplifier, the high-frequency signal generator, the step-up transformer, and the voltage multiplier are all mounted within the housing.

Alternative Implementation 23. The system of any one of Alternative Implementations 1 to 22, further comprising a controller disposed within the housing, the controller configured to control operations of the electrical circuit.

Alternative Implementation 24. The system of Alternative Implementation 23, further comprising a sensor coupled to the output element, the sensor being configured to generate data indicative of external contact with the output element, the controller configured to detect the external contact with the output element based at least in part on the data.

Alternative Implementation 25. The system of Alternative Implementation 24, wherein the controller is configured to cause the electrical circuit to generate the electrical signal in response to detecting the external contact with the output element.

Alternative Implementation 26. The system of Alternative Implementation 24, wherein the controller is configured to cause the electrical circuit to generate the electrical signal a predetermined amount of time after detecting the external contact with the output element.

Alternative Implementation 27. The system of any one of Alternative Implementations 1 to 26, wherein the output element is a generally flat glass plate, and wherein the system further comprises a conducting plate in contact with the glass plate, the conducting plate configured to receive the electrical signal from the electrical circuit and distribute the electrical signal to the glass plate.

Alternative Implementation 28. The system of any one of Alternative Implementations 1 to 27, wherein the housing forms a box and the output element includes a generally flat surface.

Alternative Implementation 29. The system of any one of Alternative Implementations 1 to 28, wherein the housing forms a wand with a grip portion that is graspable by a user, and an elongated rod extending from the grip portion, the output element being mounted at an end of the elongated rod.

Alternative Implementation 30. The system of any one of Alternative Implementations 1 to 29, wherein the output element includes a conducting plate array having a plurality of conducting plates arranged in a pattern, and wherein the electrical circuit is configured to be sequentially connected to each of the plurality of conducting plates.

Alternative Implementation 31. The system of Alternative Implementation 30, wherein the plurality of conducting plates are arranged in a spiral pattern.

Alternative Implementation 32. The system of Alternative Implementation 30 or Alternative Implementation 31, wherein the electrical circuit is configured to be sequentially connected to each of the plurality of conducting plates according to a predetermined connection frequency.

Alternative Implementation 33. The system of Alternative Implementation 32, wherein the connection frequency is less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

Alternative Implementation 34. The system of Alternative Implementation 33, wherein the connection frequency is about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

Alternative Implementation 35. The system of any one of Alternative Implementations 32 to 34, wherein the connection frequency is the same as or different from the frequency of the electrical signal.

Alternative Implementation 36. The system of Alternative Implementation 30 or Alternative Implementation 31, wherein the frequency of the electrical signal is 0 Hz such that the electrical signal is a steady-state electrical signal, and wherein electrical circuit is configured to be sequentially connected to each of the plurality of conducting plates of the conducting plate array according to a connection frequency that is between about 3 Hz and about 5 Hz.

Alternative Implementation 37. A system comprising: a housing; an electrical circuit disposed at least partially within the housing, the electrical circuit being configured to generate an electrical signal; and an optical output element configured to receive the electrical signal and output an optical signal.

Alternative Implementation 38. The system of Alternative Implementation 37, wherein a frequency of the optical signal is between about 3 Hz and about 5 Hz.

Alternative Implementation 39. The system of Alternative Implementation 38, wherein the electrical signal has a frequency that is identical to the frequency of the optical signal.

Alternative Implementation 40. The system of Alternative Implementation 38, wherein the electrical signal has a frequency that is different than the frequency of the optical signal.

Alternative Implementation 41. The system of any one of Alternative Implementations 37 to 40, wherein the optical output element is a light-emitting array includes a plurality of lasers, a plurality of light-emitting diodes, or both.

Alternative Implementation 42. The system of any one of Alternative Implementations 37 to 41, further comprising a controller disposed within the housing, the controller configured to control operations of the electrical circuit, the optical output element, or both.

Alternative Implementation 43. A method comprising: generating, via a low-frequency signal generator, a first initial signal having a target frequency; generating, via an amplifier that receives the first initial signal, a second initial signal having the target frequency and an increased amperage relative to the first initial signal; generating, via a high-frequency signal generator, a carrier signal having a carrier frequency that is greater than the target frequency; generating, via a step-up transformer that receives the second initial signal and the carrier signal, an amplitude-modulated signal having a voltage of at least 100 volts, the amplitude-modulated signal having an envelope corresponding to the target frequency; and generating, via a voltage multiplier that receives the amplitude-modulated signal, an electrical signal corresponding to the envelope of the amplitude-modulated signal and having the target frequency and a voltage of at least 100 volts.

Alternative Implementation 44. The method of Alternative Implementation 43, wherein the target frequency is less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

Alternative Implementation 45. The method of Alternative Implementation 44, wherein the target frequency is about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

Alternative Implementation 46. The method of any one of Alternative Implementations 43 to 45, wherein the voltage of the envelope signal is at least 2,000 volts.

Alternative Implementation 47. The method of any one of Alternative Implementations

43 to 46, further comprising outputting the electrical signal using an output element.

Alternative Implementation 48. The method of Alternative Implementation 47, wherein the output element includes an externally accessible glass plate mounted in a housing, a glass bulb or sphere mounted on an end of a elongated rod, or both.

Alternative Implementation 49. The method of Alternative Implementation 48, wherein the low-frequency signal generator, the amplifier, the high-frequency signal generator, the step-up transformer, and the voltage multiplier are all mounted within the housing.

Alternative Implementation 50. A method comprising generating a stimulation having a frequency of less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

Alternative Implementation 51. The method of Alternative Implementation 50, wherein the frequency is about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

Alternative Implementation 52. The method of Alternative Implementation 50 or Alternative Implementation 51, further comprising outputting the stimulation using an output element.

Alternative Implementation 53. The method of any one of Alternative Implementations 49 to 52, wherein the stimulation includes an electrical signal having a voltage of at least 100 volts.

Alternative Implementation 54. The method of any one of Alternative Implementations 49 to 51, wherein the stimulation includes an optical signal output by a light-emitting array that includes a plurality of lasers, a plurality of light-emitting diodes, or both.

Alternative Implementation 55. A method comprising: generating, via an electrical circuit, an electrical signal having a voltage of at least 100 volts; and connecting the electrical circuit to an output element, such that the electrical signal is transmitted from the output element to a user in response to a portion of the user contacting the output element, wherein the electrical signal aids in stimulating cellular regeneration in the user.

Alternative Implementation 56. The method of Alternative Implementation 55, wherein the target frequency is less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

Alternative Implementation 57. The method of Alternative Implementation 56, wherein the target frequency is about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations or alternative implementations according to aspects of the present disclosure may combine any number of features from any of the implementations described herein, such as, for example, in the alternative implementations described above.

What is claimed is:

1. A system comprising:
a housing;
an electrical circuit disposed at least partially within the housing, the electrical circuit being configured to generate an electrical signal having a frequency of less than 1,000 Hz, and a voltage of at least 100 volts; and
an output element electrically coupled to the electrical circuit and configured to output the electrical signal.

2. The system of claim 1, wherein the frequency of the electrical signal is less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

3. The system of claim 2, wherein the frequency of the electrical signal is about 3.8 Hz, about 6 Hz, about 14 Hz, about 44 Hz, about 65 Hz, about 66 Hz, about 74 Hz, about 222 Hz, about 344 Hz, about 984 Hz, or any combination thereof.

4. The system of claim 1, wherein the voltage of the electrical signal is at least 2,000 volts, and wherein the electrical signal has an amperage of between 1 μA and 20 μA.

5. The system of claim 1, wherein the frequency of the electrical signal is user-selectable.

6. The system of claim 1, wherein the frequency of the electrical signal is configured to periodically alternate between a plurality of different frequencies.

7. The system of claim 6, wherein the plurality of different frequencies includes:
a first frequency between about 60 Hz and about 70 Hz, a second frequency between about 70 Hz and about 80 Hz, and a third frequency between about 980 Hz and about 990 Hz; or
a first frequency that is less than or equal to about 10 Hz, a second frequency between about 40 Hz and about 50 Hz, and a third frequency between about 60 Hz and about 70 Hz; or
a first frequency between about 10 Hz and about 20 Hz, a second frequency between about 220 Hz and about 230 Hz, and a third frequency between about 340 Hz and about 350 Hz.

8. The system of claim 1, wherein the output element includes an externally accessible glass plate mounted in the housing, a glass bulb mounted in the housing, or a glass sphere mounted in the housing.

9. The system of claim 1, wherein the electrical circuit includes:
a low-frequency signal generator configured to generate a first initial signal having a target frequency;
an amplifier electrically connected to the low-frequency signal generator, the current amplifier being configured to receive the first initial signal and generate a second initial signal having the target frequency and an increased amperage relative to the first initial signal;
a high-frequency signal generator configured to generate a carrier signal having a carrier frequency that is greater than the target frequency;
a step-up transformer electrically coupled to the current amplifier and the high-frequency signal generator, the step-up transformer being configured to receive the second initial signal and the carrier signal and output an amplitude-modulated signal having a voltage of at least 100 volts, the amplitude-modulated signal having an envelope frequency corresponding to the target frequency; and
a voltage detector configured to receive the amplitude-modulated signal and generate the electrical signal from the envelope of the amplitude-modulated signal such that the frequency of the electrical signal is equal to the target frequency.

10. The system of claim 1, further comprising a controller disposed within the housing, the controller configured to control operations of the electrical circuit.

11. The system of claim 10, further comprising a sensor coupled to the output element, the sensor being configured to generate data indicative of external contact with the output element, the controller configured to detect the external contact with the output element based at least in part on the data, controller being configured to cause the electrical circuit to generate the electrical signal in response to detecting the external contact with the output element.

12. The system of claim 1, wherein the output element is a generally flat glass plate, and wherein the system further comprises a conducting plate in contact with the glass plate, the conducting plate configured to receive the electrical signal from the electrical circuit and distribute the electrical signal to the glass plate.

13. The system of claim 1, wherein the housing forms a box and the output element includes a generally flat surface.

14. The system of claim 1, wherein the housing forms a wand with a grip portion that is graspable by a user, and an elongated rod extending from the grip portion, the output element being mounted at an end of the elongated rod.

15. The system of claim 1, wherein the output element includes a conducting plate array having a plurality of conducting plates arranged in a pattern, and wherein the electrical circuit is configured to be sequentially connected to each of the plurality of conducting plates.

16. The system of claim 15, wherein the plurality of conducting plates are arranged in a spiral pattern.

17. The system of claim 15, wherein the electrical circuit is configured to be sequentially connected to each of the plurality of conducting plates according to a predetermined connection frequency.

18. The system of claim 17, wherein the connection frequency is less than or equal to about 10 Hz, between about 3 Hz and 5 Hz, between about 10 Hz and 20 Hz, between about 40 Hz and 50 Hz, between about 60 Hz and 70 Hz, between about 70 Hz and 80 Hz, between about 220 Hz and 230 Hz, between about 340 Hz and 350 Hz, between about 980 Hz and 990 Hz, or any combination thereof.

19. The system of claim 17, wherein the connection frequency is the same as or different from the frequency of the electrical signal.

20. The system of claim 15, wherein the frequency of the electrical signal is 0 Hz such that the electrical signal is a steady-state electrical signal, and wherein electrical circuit is configured to be sequentially connected to each of the plurality of conducting plates of the conducting plate array according to a connection frequency that is between about 3 Hz and about 5 Hz.
